(19) United States
(12) Patent Application Publication
(10) Pub. No.: US 2022/0031394 A1
(43) Pub. Date: Feb. 3, 2022

(54) Method and System for Providing Real Time Surgical Site Measurements

(71) Applicant: Asensus Surgical US, Inc., Durham, NC (US)

(72) Inventors: Kevin Andrew Hufford, Cary, NC (US); Tal Nir, Haifa (IL); Mohan Nathan, Raleigh, NC (US)

(73) Assignee: Asensus Surgical US, Inc., Durham, NC (US)

(21) Appl. No.: 17/035,534

(22) Filed: Sep. 28, 2020

Related U.S. Application Data
(60) Provisional application No. 62/907,449, filed on Sep. 27, 2019.
(60) Provisional application No. 62/934,441, filed on Nov. 12, 2019.

(51) Int. Cl. A61B 34/10; G06T 7/62; G06T 7/00; G06F 3/0484; G06T 7/50; A61F 2/00; A61B 90/00; G16H 30/20

(52) U.S. Cl. CPC A61B 34/10; G06T 7/62; G06T 7/0012; G06F 3/04845; G06T 7/50; G06F 3/0482; A61B 90/361; A61B 90/37; G16H 30/20; G06T 2207/30052; G06T 2200/24; A61F 2/0063

(57) ABSTRACT

A system and method for measuring an area of interest such as a hernia defect within a body cavity, in which real time image data is captured at a treatment site that includes the area of interest. Computer vision is applied to identify the extents of the area of interest within images captured using the camera, and dimensions of the area of interest are measured using the image data. The user is given output, which may include recommendations of hernia mesh shape/size and or positioning, based on the measured dimensions.

This application claims the benefit of U.S. Provisional Application No. 62/907,449, filed Sep. 27, 2019, and U.S. Provisional Application No. 62/934,441, filed Nov. 12, 2019, each of which is incorporated herein by reference.

### BACKGROUND

There are various contexts in which it is useful for a practitioner performing surgery to obtain area and/or depth measurements for areas or features of interest within the surgical field.

One context is that of hernia repair. After closure of a hernia, a surgical mesh is often inserted and attached (via suture or other means) to provide additional structural stability to the site and minimize the likelihood of recurrence. It is important to size this mesh correctly, with full coverage of the site along with adequate margin provided along the perimeter to allow for attachment to healthy tissue—distributing the load as well as minimizing the likelihood of tearing through more fragile tissue at the boundaries of the now-closed hernia.

The size of the area to be covered and thus the size of the mesh needed may currently be estimated by a user looking at the endoscopic view of the site. For example, the user might use the known diameters or feature lengths on surgical instruments as size cues. In more complex cases, a sterile, flexible, measuring “tape” may be rolled up, inserted through a trocar, unrolled in the surgical field, and manipulated using the laparoscopic instruments to make the necessary measurements.

This application describes a system providing more accurate sizing and area measurement information than can be achieved using current methods.

### BRIEF DESCRIPTION OF THE DRAWINGS

FIG. 1 is a block diagram schematically illustrating a system according to the disclosed embodiments.

FIGS. 2-11 illustrate steps of one example of a method for providing sizing information for surgical mesh using concepts described in this application. More particularly,

FIG. 2 illustrates an endoscopic display during placement, using input from a user, of a graphical boundary around a hernia captured in the endoscopic image.

FIG. 3 is similar to FIG. 2, and further shows the graphical boundary shifted over a greater portion of the defect and beginning to be expanded in response to user input.

FIG. 4 is similar to FIG. 3, and shows the graphical boundary expanded to fully encircle the defect.

FIG. 5 illustrates initiation of the use of an active contour model to identify the perimeter of the hernia in the endoscope image;

FIG. 6 further illustrates further process of the active contour model towards identifying the perimeter of the hernia in the endoscopic image;

FIG. 7 shows the perimeter once it has been fully-identified using the active contour model;

FIGS. 8 and 9 are similar to FIG. 7, but additionally shows overlays depicting margins of 0.5cm and 0.7cm, respectively, around the determined perimeter.

FIG. 10 shows an overlay of dimensions matching those of a recommended mesh size overlaid on the image of the defect and conforming to the tissue topography.

FIG. 11 illustrates a sequence of steps following in the Example 1 method of using the system.

FIGS. 12 and 13 illustrate alternative ways in which sizing information may be overlaid onto the image of the hernia.

FIG. 14 illustrates an image of a defect detected using an active contour model and illustrates use of depth disparities to confirm boundaries or measurements derived based on the active contour model.

FIG. 15 illustrates an image of a defect with lines A and B crossing the image of the defect, and further shows cross-sections of the defect along lines A and B to illustrate use of a mesh model having sufficient tension so that the mesh displayed as in FIG. 10 bridges the recess of the defect.

FIG. 16 illustrates a sequence of steps following in the Example 2 method of using the system.

FIG. 17A shows an example of an image display of a defect, with available mesh size/shape options shown on the image display.

FIG. 17B is similar to FIG. 17A but shows the display after one of the available mesh options has been selected and positioned as an overlay over the displayed defect.

FIG. 17C is similar to FIG. 17B but shows a different one of the available mesh options selected and overlaid.

### DETAILED DESCRIPTION

This application describes a system and method that use image processing of the endoscopic view to determine sizing and measurement information for a hernia defect or other area of interest within a surgical site.

Examples of ways in which an area in a surgical field may be measured are described here, but it should be understood that others may be used without deviating from the scope of the invention. Additionally, examples are given in this application in the context of hernia repair, but the disclosed features and steps are equally useful for other clinical applications requiring measurement of an area of interest within the surgical site and, optionally, selection of an appropriately-sized implant or other medical device for use at that site.

System

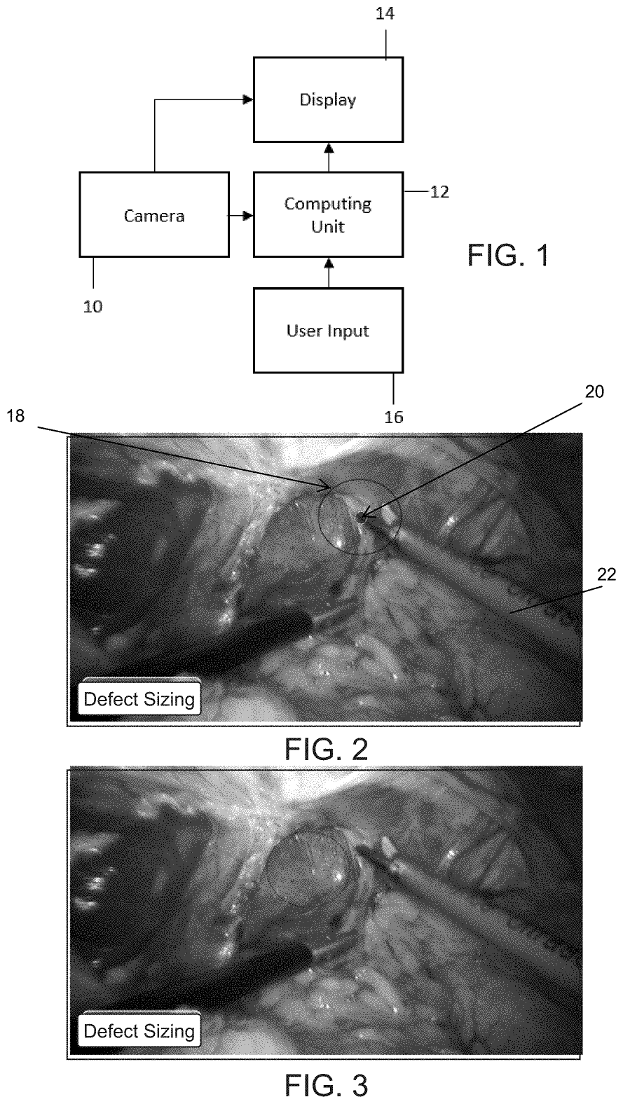

A system useful for performing the disclosed methods, as depicted in FIG. 1, may comprise a camera 10, a computing unit 12, a display 14, and, preferably, one or more user input devices 16.

The camera 10 may be a 3D or 2D endoscopic or laparoscopic camera. Where it is desirable to obtain depth measurements or determination of depth variations, configurations allowing such measurements (e.g. a stereo/3D camera, or a 2D camera with software and/or hardware configured to permit depth information to be determined or derived) are used. The computing unit 12 is configured to receive the images/video from the camera and input from the user input device(s). An algorithm stored in memory accessible by the computing unit is executable to, depending on the particular application, use the image data to perform one or more of the following (a) image segmentation, such as for identifying boundaries of an area of interest that is to be measured; (b) recognition of hernia defects or other predetermined types of areas of interest, based on machine learning or neural networks; (c) point to point measurement; (d) area measurement; and (e) computing the depth (if not done by the camera itself), i.e. the distance between the image sensor and the scene points captured by the image, which in the case of a laparoscope or endoscope are points within a body cavity using data from the camera. The computing unit may also include an algorithm for generating overlays to be displayed on the display.

The system may include one or more user input devices 16. When included, a variety of different types of user input devices may be used alone or in combination. Examples include, but are not limited to, eye tracking devices, head tracking devices, touch screen displays, mouse-type devices, voice input devices, foot pedals, or switches. Various movements of an input handle used to direct movement of a component of a surgical robotic system may be received as input (e.g. handle manipulation, joystick, finger wheel or knob, touch surface, button press). Another form of input may include manual or robotic manipulation of a surgical instrument having a tip or other part that is tracked using image processing methods when the system is in an input-delivering mode, so that it may function as a mouse, pointer and/or stylus when moved in the imaging field, etc. Input devices of the types listed are often used in combination with a second, confirmatory, form of input device allowing the user to enter or confirm (e.g. a switch, voice input device, button, icon to press on a touch screen, etc., as non-limiting examples).

The following steps may be carried out when using the disclosed system:

Analysis of a surgical site in real time using computer vision

In an initial step, image processing techniques are used in real time on images of the surgical site to identify the area to be measured. Embodiments for carrying out this step include, without limitation, the following:

(a) a system configured so that any hernia defects or other areas of interest (lesions, organs, tumors etc.) captured in the endoscopic images are automatically detected by the image processing system. In some forms of this embodiment, a machine learning algorithm such as, for example, one utilizing neural networks analyzes the images and detects the defects or other predetermined items of interest. In some embodiments, color variations and/or depth disparities (see the section entitled Depth Disparities below) are detected in order to locate the defect. The system may generate feedback to the user that calls detected areas of interest or defects to the attention of the user, by, for example, displaying a graphical marking (e.g. a perimeter around the area of interest, such as the region in which the defect is located, or a color or textured overlay on the region in which the defect is located) and/or text overlay on the image display. The user may optionally be prompted to confirm using a user input device that an identified area is a hernia defect that should be measured.

(b) a system configured to receive user input identifying a region within which a hernia defect or other area of interest is located. For example, while observing the image on the image display, the user places or draws a perimeter around the region within which the defect or area of interest is located. In this example, it is desirable, but optional, that the system generate and display a graphical marking corresponding to the input being given by the user. The graphical marking may correspond to the shape “drawn” by the user using the user interface, or it may be a predetermined shape (e.g. oval, circle, rectangle) that the user places overlaying the defect site on the displayed image and drags to expand/contract the shape to fully enclose the defect. Suitable input devices for this configuration include a manually- or robotically-manipulated instrument tip moved within the surgical field as a mouse or pen while it is tracked using a computer vision algorithm to create the perimeter, a user input handle of a surgeon console of a robotic system operated as a mouse to move a graphical pointer or other icon on the image display (optionally with the robotic manipulators or instruments, as applicable, operatively disengaged or “clutched” from the user input so as to remain stationary during the use of the handles for mouse-type input) or a finger or stylus on a touch screen interface. The system is programmed so that once the input is received, the system can identify the area of interest defect using algorithms such as those described above.

(c) a system configured to receive user input identifying points between which measurements should be taken and/or an area to be measured. In these embodiments, rather than identifying the hernia defect or other area of interest using image processing, image processing is used to receive input from the user corresponding to points between which measurements are to be taken or areas that are to be measured. More specifically, image processing techniques are used to record the locations or movements of instrument tips or other physical markers positioned by a user in the operative site to identify to the system points between which measurements are to be taken, or to circumscribe areas that are to be measured. As one specific example, the user places the tip(s) to identify to the system points between which measurements should be taken, and image processing is used to recognize the tip(s) within the image display. In this embodiment, the user might place two or more instrument tips at desired points at the treatment site between which measurements are desired and prompt the system to determine the measurements between the instrument tips, or between icons displayed adjacent to the tips. Alternatively, the user might move an instrument tip to a first point and then to a second point and prompt the system to then determine the distances between pairs of points, with the process repeated until the desired area has been measured. Graphical icons or pins may be overlayed by the system at the locations on the display corresponding to those identified by the user as points to be used as reference points for measurements.

As another specific example, the user might circumscribe an area using multiple points or an area “drawn” using the instrument tip and prompt the system to measure the circumscribed area. In this example, the user could trace the perimeter of the defect or other object or area of interest. The steps are repeated as needed to obtain the dimensions for the desired area. Note that when measurement techniques are used in a system employing robotically-manipulated instruments, kinematic information may be used to aid in defining the location of the instrument tips in addition to, or as an alternative to, the use of image processing.

Measurement of a hernia site or other area of interest—Measurement may be carried out in a variety of ways, including using 2D and 3D measurement techniques, many of which are known to those skilled in the art. In preferred embodiments, 3D measurement techniques are used to ensure optimal measurement accuracy. The “Example” section of this application includes additional information concerning measurement techniques that may be used.

Dimensions for a hernia mesh provided to the user. When the system is used as a tool for determining the size of a suitable mesh for the defect, the dimensions may be provided in the form of the dimensions of a size of mesh to be prepared for implantation, or the selection of one of a fixed number of mesh sizes available for implantation, or some other output enabling the user to choose the mesh size or size and shape suitable for the hernia defect. In other examples, overlays of mesh shapes in a selection of sizes may be displayed on the display (scaled to match the scale of the displayed image), allowing the user to visually assess their suitability for the defect site.

In some implementations, the system may take the measured dimensions and automatically add a safe margin around its perimeter. In these cases, the system may propose a corresponding mesh size and shape that covers the defect plus the margin. The width of the margin may be predefined or entered/selected by the user using an input device. The perimeter of this mesh may be adjusted by the user.

This system may be used during laparoscopic or other types of surgical procedures performed with manual instruments, or in a robotically-assisted procedures where the instruments are electromechanically maneuvered or articulated. It may also be used in semi- or fully-autonomous robotic surgical procedures. Where the system is used in conjunction with a surgical robotic system, the enhanced accuracy, user interface, and kinematic information (e.g. kinematic information relating to the location of instrument tips being used to identify sites at which measurements are to be taken) may increase the accuracy of the measurements and provide a more seamless user experience.

Some specific examples of use of the described system will now be given. Each of the listed examples may incorporate any of the features or functions described above in the “System” section.

### EXAMPLE 1

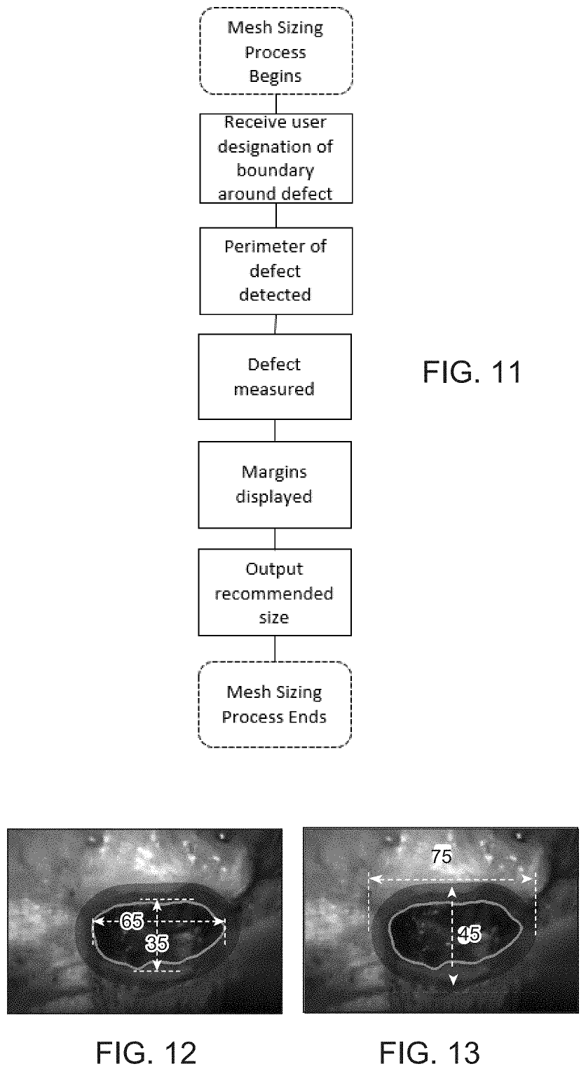

FIGS. 2-10 depict a display of an endoscopic image of a hernia site, and illustrate the steps, shown in the block diagram of FIG. 11, of a first exemplary method for using the concepts described in this application. If the hernia is to be sutured closed before application of the mesh, this method might be performed before or after suturing. FIGS. 2-10 illustrate sizing of a defect that has not been sutured before the defect sizing operation.

In this example, an image of the operative site is captured by an endoscope and displayed on a display. See FIG. 2. The user may give a command to the system to enter a defect sizing mode. A graphical overlay may be displayed confirming that the system has entered that mode. A user viewing the image on the display designates a boundary around the defect by placing or drawing a border 18 (FIG. 4) surrounding the defect as displayed on the display. The system causes this border to appear as an overlay on the display.

As shown in FIG. 2, in one specific embodiment placement of the border may begin with the system marking a point 20 adjacent to the tip of a surgical instrument 22 positioned at the defect site (e.g. at an edge or some other part of the defect site), and placing the border 18 surrounding the point 20. In the figures the border is shown as a circle, but it may have any regular or irregular shape. The user can reposition (FIG. 3) and expand (FIG. 4) the border (or, in other embodiments, “draw” it on the display) by moving the tip of an instrument 22 within the operative site. During placement or drawing of the border, the instrument tip location is recorded by the system using image processing and/or kinematic methods. Alternative forms of user input that may be used to place the border are described in the “System” section above.

In other embodiments, the image processing algorithm automatically detects the defect, and expands and automatically repositions the border 18 to surround it, optimally then receiving user confirmation using a user input device that the defect has been encircled.

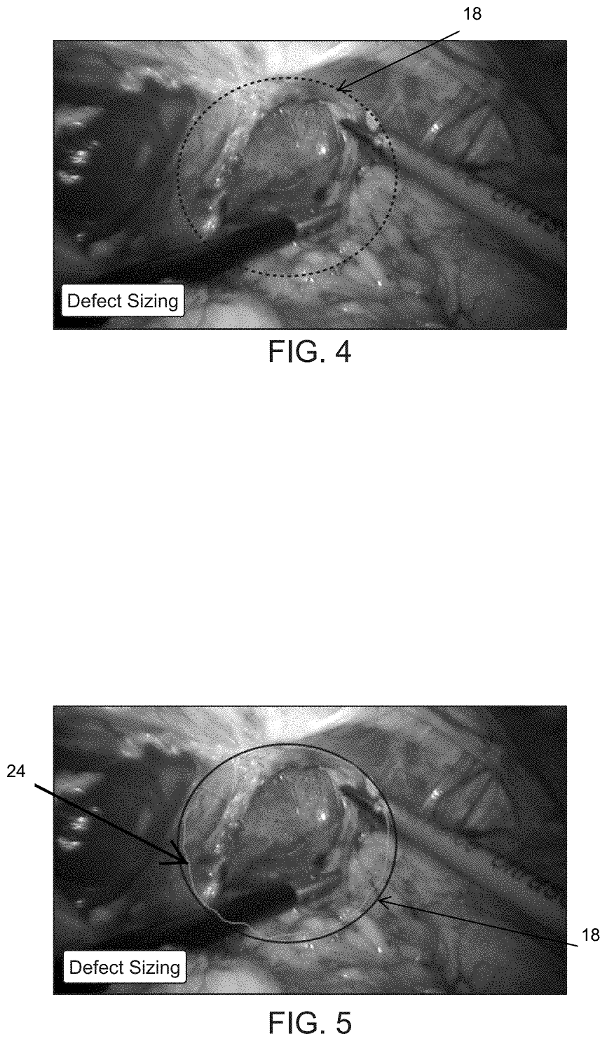

Once the user has identified the region within which the area of interest or defect is located, a computer vision algorithm is employed to determine the boundaries of the area of interest or defect. Various techniques for carrying out this process are described above in (a). In this specific example, to detect the perimeter of the detect, the system places an active contour model 24 within the border placed or confirmed by the user, as shown in FIG. 5, and begins to shrink the active contour model towards the physical perimeter of the hernia. During use of the active contour model, the physical perimeter or “edge” of the hernia is “seen” by the image processing system using color differences (and/or differences in brightness) between pixels of the area inside and the area outside the perimeter, and/or (where a 3D system is used) using depth differences between the area inside and the area outside the perimeter. For additional details on this later concept, see the section below entitled “Depth Disparities.” The active contour model is preferably (but optionally) shown on the image display so that, upon completion, the user can visually confirm that it has accurately identified the border.

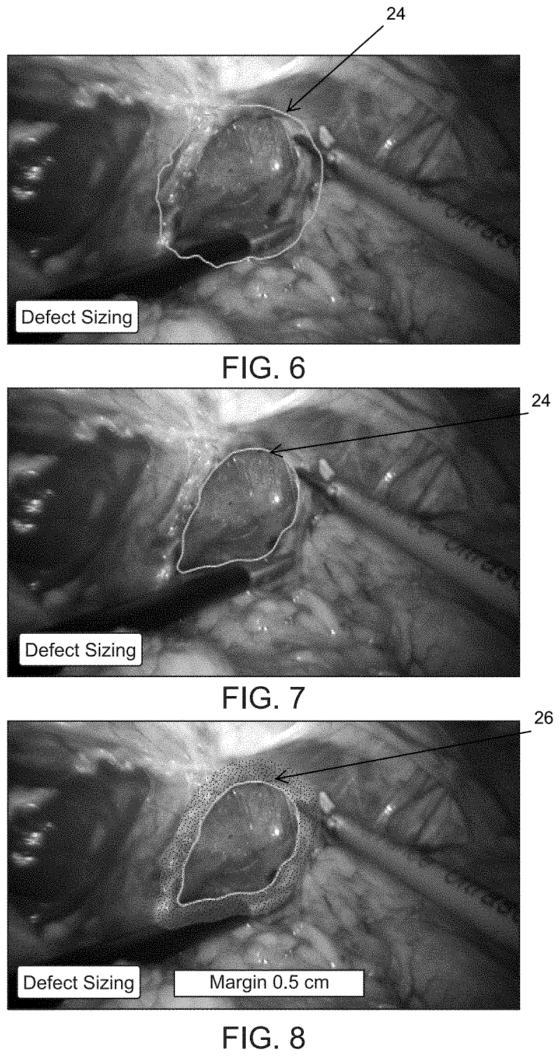

FIG. 6 shows the highlighted contour model beginning to form around the perimeter of the hernia defect. The computer vision/active contour model detects the edges of the defect and stops shrinking a portion of the model once that portion contacts an edge in a certain region, while the rest of the model also shrinks until it, too, contacts an edge. This process continues until the entire perimeter of the defect is identified by the active contour model, as shown in FIG. 7. The user may optionally be prompted to confirm, using input to the system, that the perimeter appropriately matches the perimeter of the hernia.

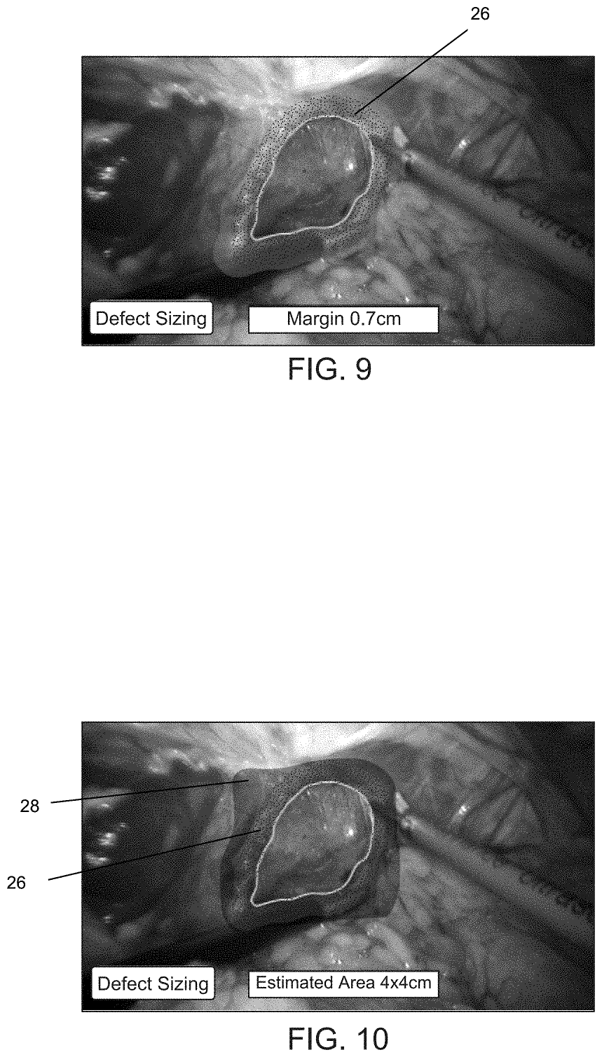

Before or after measuring the defect, the system may display a margin overlay 26 on the image display, around the perimeter of the defect. This overlay has an outer edge that runs parallel to the edge of the defect, with the width of the overlay corresponding to a predetermined margin around the defect. In FIG. 8 a margin of 0.5 cm is shown displayed, and in FIG. 9 a margin of 0.7 cm is shown. The particular sizes of the margins may be programmed into the system and selected by the user from a menu or specified by the user using an input device.

The user inputs instructions to the system confirming the selected margin width. The system measures the dimensions and, optionally the area, of the hernia, preferably using 3D image processing techniques as described above. The system measures the largest dimensions of the defect based on the perimeter defined using the active contour model. The nature of the measurement may include measurement across the defect from various portions of its edge to determine the largest dimensions in perpendicular directions across the defect. If a circular mesh is intended, the largest dimension in a single direction across the defect may be measured.

A recommended mesh profile 28 and/or recommended mesh dimensions are overlaid onto the image. Where the user has specified the margin width, or the system is programmed to include a predetermined margin width, the recommended profile is preferably a shape having borders that surround the defect by an amount that creates at least the chosen or predetermined margin around the defect. In FIG. 10, a rectangular overlay 28 corresponding to a best rectangular fit to the defect size and margin has been generated by the system and displayed, together with the recommended dimensions for a rectangular piece of mesh for the hernia. The system displays the overlay with a scale selected to match the scale of the displayed image of the defect (as determined through one or more of camera calibration by the system, input to the system from the camera indicating the real-time digital or optical zoom state of the camera, input to the system of kinematic information from a robotic manipulator carrying the camera, etc.) so that the size of the mesh overlay will be in proportion to the size of the defect. Because the tissue topography at the defect site is known, the overlay depiction of the mesh is shown as it would appear if secured in place, following the contours of the underlying tissue, except for the deeper recess of the defect itself, as discussed in greater detail in the section below entitled “Depth Disparities.” The margin 26 is also optionally displayed.

The displayed overlay, as well as others described in this application, is preferably at least partially transparent so as to not obscure the user's view of the operative site. The user may wish to choose the position and/or orientation for the mesh, or to deviate from the algorithm-proposed position and/or orientation, if for example, the user wants to choose certain robust tissue structures as attachment sites and/or to choose the desired distribution of mesh tension. The system thus may be configured to receive input from the user to select or change the orientation of the displayed mesh. For example, the user may give input to drag and/or rotate the mesh overlay relative to the image. As another example, the system may automatically, or be prompted to, identify the primary and secondary axes of the defect, and automatically rotate and skew a displayed rectangular or oval shaped mesh overly to align its primary and second axes with those of the defect. The user may from this point use the user input device to fine tune the position and orientation.

Note that the measurement techniques may be used to measure the defect itself (based on the perimeter defined using the active contour model) and to output those measurements to the user as depicted in FIG. 12, or to calculate and output dimensions of the recommended mesh profile (the defect size plus the desired margin) as shown in FIG. 13, or to calculate and output the dimensions of a rectangle or other shape fit to the recommended mesh profile (in each case preferably using 3D techniques to account for depth variations) as discussed in connection with FIG. 10.

In modifications to Example 1, neural networks may be trained to recognize hernia defects, and/or to identify optimal mesh placement and sizing.

In another modification to Example 1, rather than encircling an area, a user input device is used to move a cursor (crosshairs) or other graphical overlay to define a point inside a defect or region to be measured as it is displayed in real time on the display. A region growing algorithm is then executed, expanding an area from within that point by finding within the image data continuity of color or other features within some tolerance that are used to identify the extents of the area of interest.

Depth Disparities

As discussed in connection with Example 1, segmentation methods often use color differentiation or edge detection methods to determine the extent of a given region, such as the hernia defect. In certain instances, the color information may change across a region, creating potential for errors in segmentation and therefore measurement. It can therefore be beneficial to enrich the fidelity of segmentation and classification of regions by also using depth information, which may be gathered from a stereo endoscopic camera. Using detection of depth disparities, significant changes in depth across the region identified as being the defect can be used by the system to confirm that the active contour model detection of edges is correct.

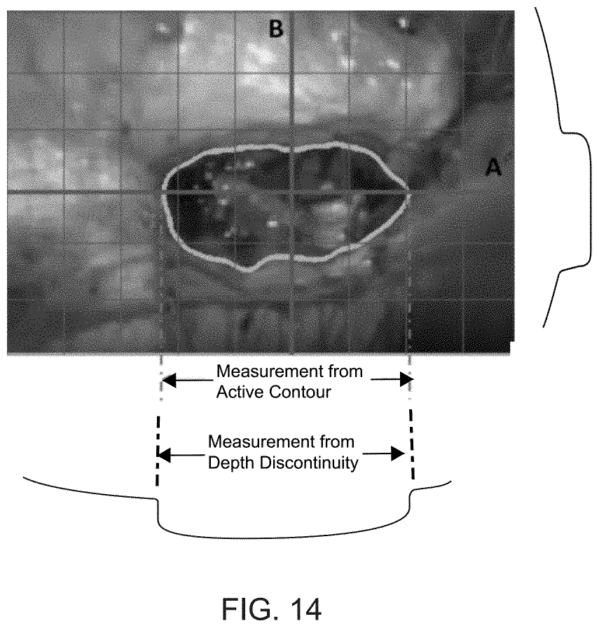

FIG. 14 illustrates the defect from Example 1, with the detected perimeter highlighted, and with horizontal and vertical lines A and B shown crossing the defect. To the right of the image is a cross-section view of the defect site taken along a plane that extends along line B and is perpendicular to the plane of the image. Below the image is a cross-section view of the defect site taken along a plane that extends along line A and runs perpendicular to the plane of the image.

This illustrates that the extents of the defect as defined using color edge detection along lines A and B match those defined using depth disparity detection.

In use, during the edge identification process, the depth disparity information can be used as illustrated in FIG. 14 to check the accuracy of the edge detection information by measuring depth variations across various lines crossing the field of view, and comparing those with measurements taken along those lines between edges detected using color edge detection. If the measurements obtained using edge detection are within a predetermined margin of error compared with those obtained using depth disparities, the measurements are confirmed for display to the user or use in guiding mesh selection as described. Alternatively, the system can be configured to, on determining which pixels or groups of pixels in the captured images identify edges using color differentiation or other edge detection techniques, determine which of those pixels or pixel groups are in close proximity to detected depth disparities of above a predetermined threshold (e.g. in excess of a predetermined change in depth over a predetermined distance along the reference axis). Those that are will be confirmed to accurately identify edges of the defect and may be used as the basis for measurements and other actions described in this application. Color differentiation and depth disparity analysis can instead be performed simultaneously, with pixels or groups of pixels that predict the presence of an edge using both color differentiation and depth disparity techniques being identified as those through which an edge of the defect passes and then used as the basis for measurements and other actions described in this application.

As another example, a user might use a user input device to place overlays of horizontal and vertical lines or crosshairs within the defect as observed on the image display. These lines could be used to define horizontal and vertical section lines along which depth disparities would be sought. Once found, the defects could be traced circumferentially to define the maximum extent of the area/region/defect, and the measurements would be taken from those extents.

It is not required that depth disparity detection be used in combination with, or as a check, on edge detection carried out using active contour models. It is a technique that may be used on its own for edge detection, or in combination with other methods such as machine learning/neural networks.

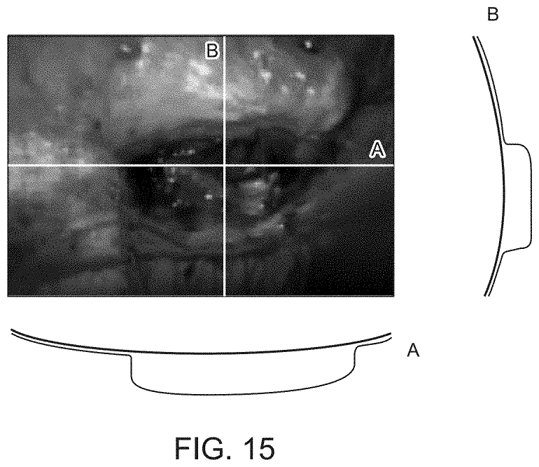

Referring to FIG. 15, detection of depth disparities may also be used when a proposed position and orientation of a mesh is displayed as an overlay. As discussed in connection with FIG. 10, the displayed mesh preferably is displayed to follow the topography of the tissue surrounding the defect, so that the user can see an approximation of where the edges of the mesh will position on the tissue. However, because the mesh will not be pressed into the recess of the defect, it is desirable to display the mesh overlay as it would be implanted—i.e. to display it so that it does not follow into that recess, but instead bridges the recess as shown in FIG. 15. The system may therefore be programmed to maintain a predetermined level of “tension” in the mesh model, so that it follows the contours of the tissue located around the defect but does not significantly increase its path length by following the deep contour of the recess.

### EXAMPLE 2

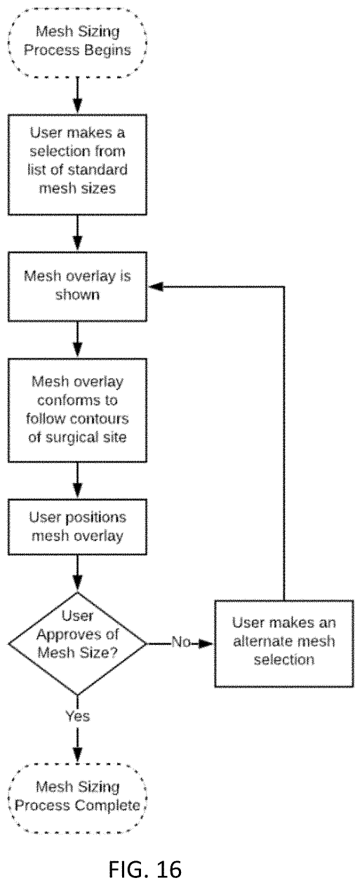

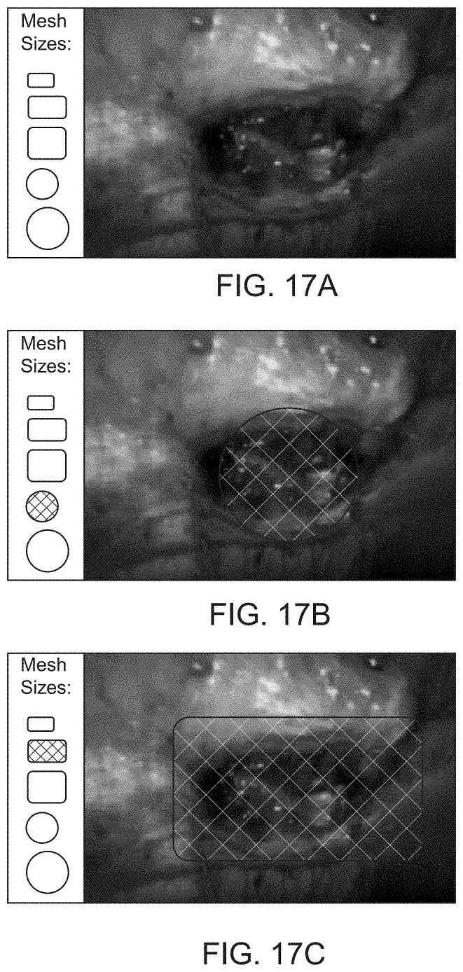

In a second example depicted in FIG. 16, mesh overlays corresponding to sizes available for implantation (such as standard commercially available sizes) are displayed to the user on the image display that is also displaying the operative site. For example, a collection of available shapes and sizes may be simultaneously displayed on the image display as shown in FIG. 17A. While not shown in FIG. 17A, text indicating dimensions or other identifying information for each mesh type may be displayed with each overlay.

In this embodiment, the system may be configured to detect the defect as described with Example 1. Alternatively, the system may be configured to determine 3D surface topography but to not necessarily determine the edges of the defect.

User input is received by which the user “selects” a first one of the displayed mesh types. As one specific example, the user may rotate a finger wheel or knob on the user input device to sequentially highlight each of the displayed mesh types, then give a confirmatory form of input such as a button press to confirm selection of the highlighted mesh. Once confirmed, the system displays the selected mesh type in position over the defect (if the edges of the defect have been determined by the system), or the user gives input to “pick up” and “drag” the selected mesh type into a desired position over the defect. The system conforms the displayed mesh overlay to the surface topography, while maintaining tension across the defect, as discussed in connection with Example 1. See FIG. 17B. The user may then optionally choose to reposition or reorient the overlay as also discussed in the description of Example 1. To evaluate a second one of the mesh types, the user gives input “selecting” a second mesh type and the process described above is repeated to position the second mesh type overlayed on the defect. See FIG. 17C. In this step the first mesh type may be automatically removed as an overlay on the defect, actively removed by the user using an instruction to the system to remove it, or left in place so that the first and second mesh types are simultaneously displayed (optionally using different colors or patterns) to allow the user to directly compare the coverage provided by each.

### EXAMPLE 3

In this embodiment, the system is configured to detect the defect as described with Example 1, and the method is performed similarly to Example 1, with a recommended mesh size and orientation displayed as in FIG. 10. The system next receives input from the user to change the overlay. The change may be to increase or decrease the size of the displayed mesh. For example, the first displayed mesh may be one of a plurality of predetermined sizes available for implantation (such as standard commercially available sizes), and the input may be to change the displayed mesh to match the size and shape of a second one of those sizes, etc. As another example, the change may be to replace the displayed mesh with a second one of the available mesh shapes/sizes. The mesh options may optionally display on screen as depicted in FIGS. 17A-17C, with the mesh disposed on the overlay at any given time highlighted using a color, pattern, etc. or other visual marking as in FIG. 17B.

### EXAMPLE 4

In this embodiment, the system is configured to detect the defect as described with Example 1, and the method is performed similarly to Example 1. Once the defect is detected, all available mesh types are simultaneously displayed on the defect, each with coloring to differentiate it from the other displayed mesh overlays (e.g. different color shading and/or border types, different patterns, etc.). Each overlay is oriented as determined by the system to best cover the defect given the size and shape of the defect and the size and shape of the corresponding mesh, and to conform to the topography but with tension across the defect as described in the prior examples. Further user input can be given to select and re-position displayed mesh overlays as discussed with prior examples, and to remove mesh types that have been ruled out from the display.

We claim:

1. A system for aiding selection of a hernia mesh for treating a hernia defect, comprising:
a camera positionable to capture image data corresponding to a treatment site that includes a hernia defect;
at least one processor and at least one memory, the at least one memory storing instructions executable by said at least one processor to:
identify at least a portion of the hernia defect within images captured using the camera;
measure a dimension relating to the hernia defect based on the image data; and
provide output to a user based on the measured dimension.

2. The system of claim 1, wherein the instructions are further executable by said at least one processor to determine dimensions of a recommended mesh implant for covering the defect, and wherein the output includes signals to generate a display of the dimensions on an image display.

3. The system of claim 2, wherein the output includes text describing measured dimension.

4. The system of claim 2, wherein the output includes a display of an overlay indicating the boundaries of the determined dimensions overlaying the hernia defect on the display.

5. The system of claim 4, wherein the instructions are further executable by said at least one processor to determine variations in topography of tissue at the treatment site, and to display the overlay to conform to the variations in topography.

6. The system of claim 5, wherein the instructions are further executable by said at least one processor to display the overlay to maintain tension across depth disparities exceeding a predetermined change in depth.

7. The system of claim 1, further including a user input, wherein the instructions are further executable by said at least one processor to move or rotate the position of the overlay relative to the displayed image in response to input from the user input.

8. The system of claim 1, wherein the instructions are executable by said at least one processor to:
display a plurality of overlays on an image display, each representing a different mesh implant of a predetermined size and shape;
in response to user input, positioning a select one of the overlays over the hernia defect displayed on the image display;
determine variations in topography of tissue at the treatment site, and to display the select one of the overlays to conform to the variations in topography.

9. A method for aiding selection of a hernia mesh for treating a hernia defect, comprising the steps of:
capturing image data corresponding to a treatment site that includes a hernia defect;
using computer vision to identify at least a portion of the hernia defect within images captured using the camera;
measuring a dimension relating to the hernia defect based on the image data; and
providing output to a user based on the measured dimension.

10. The method of claim 9, further including determining dimensions of a recommended mesh implant for covering the defect, and wherein the output includes signals to generate a display of the dimensions on an image display.

11. The method of claim 10, wherein the determining steps includes determining dimensions that will cover the defect with a predetermined margin width.

12. The method of claim 11, wherein the method includes receiving user input selecting the predetermined margin width.

13. The method of claim 10 wherein the output includes a display of an overlay indicating the boundaries of the determined dimensions overlaying the hernia defect on the display.

14. The method of claim 10, further including using the image data to determine variations in topography of tissue at the treatment site, and to display the overlay to conform to the variations in topography.

15. The method of claim 14, further including displaying the overlay to maintain tension across depth disparities exceeding a predetermined change in depth.

16. The method of claim 1, further including changing the position or orientation of the overlay relative to the displayed image in response to user input.

17. The method of claim 1, further including:
displaying a plurality of overlays on an image display, each representing a different mesh implant of a predetermined size and shape;
in response to user input, positioning a select one of the overlays over the hernia defect displayed on the image display;
determining variations in topography of tissue at the treatment site using the image data, and
displaying the select one of the overlays to conform to the variations in topography.

18. A method of measuring an area of interest within a body cavity, comprising the steps of:
capturing image data corresponding to a treatment site that includes the area of interest;
using computer vision to identify the extents of the area of interest within images captured using the camera;
measuring a dimension relating to the area of interest based on the image data; and
providing output to a user based on the measured dimension.

19. The method of claim 18, further including:
receiving user input defining a boundary encircling the area of interest as displayed on the image display; and
applying computer vision within the encircled area to identify the extends of the area of interest within the images.

20. The method of claim 18, wherein the measured dimension is at least one of length, width, depth, or area.
